(19) United States
(12) Patent Application Publication
(10) Pub. No.: US 2019/0038691 A1
(43) Pub. Date: Feb. 7, 2019

(54) METHOD FOR CONVERSION AND EXTRACTION OF BENEFICIAL COMPONENTS IN GINSENG

(71) Applicant: EGEONJO.CO.,LTD, Jeju-si (KR)

(72) Inventor: Ken Lee, Jeju-si (KR)

(21) Appl. No.: 15/814,552

(22) Filed: Nov. 16, 2017

(30) Foreign Application Priority Data
Aug. 3, 2017 (KR) 10-2017-0098609

(51) Int. Cl. A61K 36/258; A61K 31/702

(52) U.S. Cl. CPC A61K 36/258; A61K 31/702; A61K 2236/51; A61K 2236/37; A61K 2236/17

(57) ABSTRACT

Provided is a method for conversion and extraction of beneficial components, the method including: a vacuum step for feeding ginseng into a vacuum chamber and applying a vacuum pressure thereinto; and a treatment step for heating and drying the ginseng under the vacuum pressure, so that the loss of beneficial components can be minimized and the treatment cost can be lowered by simplifying the treatment procedure and shortening the treatment time, and the saponin conversion and extraction efficiencies can be effectively increased through a simple treatment procedure.

### CROSS-REFERENCE TO RELATED APPLICATION

This application claims priority to and the benefit of Korean Patent Application No. 10-2017-0098609 filed in the Korean Intellectual Property Office on Aug. 3, 2017, the entire contents of which are incorporated herein by reference.

### BACKGROUND OF THE INVENTION

### (a) Field of the Invention

Disclosed is a method for conversion and extraction of beneficial components in ginseng, the method being capable of easily converting and effectively extracting beneficial components in ginseng.

### (b) Description of the Related Art

Ginseng contains various beneficial components, such as several kinds of saponins (approximately 30 kinds of ginsenosides), nitrogen-containing compounds (proteins, amino acids, alkaloids, etc.), phenols, and lipids, and thus can be favorably used as a health supplement food. In recent years, ginseng has been increasingly used not only as a health supplement food but also as a raw material for cosmetics and the like.

It is known that ginsenosides having high molecular weights are converted into ginsenosides having low molecular weights during high-temperature treatment of ginseng. Conventionally, in order to utilize this phenomenon, it was a conventional practice to induce changes in constitutional components by repeating, several times, a method of steaming and drying at a high temperature and a high pressure. That is, conventionally, the kind of saponin was verified through a procedure of steaming or drying ginseng.

However, such a conventional method was very costly, and increased the loss of beneficial components in ginseng itself during repeated steaming, causing a reduced yield.

Moreover, since ginseng was heated at a high temperature, the tissues of ginseng may be carbonized to produce toxic materials, such as dioxins, if the conditions are not appropriate.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a method for conversion and extraction of beneficial components in ginseng, the method having advantages of minimizing the loss of beneficial components and lowering the treatment cost by simplifying the treatment procedure and shortening the treatment time.

The present invention has been made in an effort to provide a method for conversion and extraction of beneficial components in ginseng, the method having advantages of effectively increasing saponin conversion and extraction efficiencies through a simple treatment procedure.

An exemplary embodiment of the present invention provides a method for conversion and extraction of beneficial components in ginseng, the method including: a vacuum step of feeding ginseng into a vacuum chamber and applying a vacuum pressure thereinto; and a treatment step of heating and drying the ginseng under the vacuum pressure.

In the vacuum step, the vacuum pressure may be −95 kPa to −98 kPa.

In the treatment step, the temperature for the heating may be 60° C. to 120° C. The treatment step may be carried out for 5 hours to 24 hours.

The method may further include a collecting step of condensing and collecting moisture generated during the drying of the ginseng in the treatment step.

According to the present embodiment as described above, the treatment time is drastically shortened to minimize the loss of beneficial components and reduce the cost required for treatment, thereby increasing cost competitiveness.

Furthermore, the change of the ginseng composition can be induced through short-time treatment, and saponins can be effectively converted and extracted through a simple treatment procedure, thereby obtaining an effect of increasing the extraction amounts of saponins per unit weight of dried ginseng.

Furthermore, the composition of materials contained in the steam evaporating during the treatment procedure can be changed to increase the beneficial effect of an extract.

Furthermore, the evaporation of beneficial components can be induced even at a low temperature through the vacuum environment, and the beneficial components can be collected and used.

Furthermore, the treatment conditions can be easily adjusted to induce a difference in the ginseng components so as to be suitable for the purpose of use, thereby producing various beneficial components.

Furthermore, the present invention can be applied to various plants, such as Chamaecyparis obtusa and Phellodendron amurense, including balloon flowers, Codonopsis lanceolata, burdock, and the like, which have high saponin contents, besides ginseng, thereby increasing the extraction efficiency and sensory quality of beneficial components in various crops.

### BRIEF DESCRIPTION OF THE DRAWING

FIG. 1 is a schematic diagram showing a structure of a facility for conversion and extraction of beneficial components in ginseng according to the present embodiment.

### DETAILED DESCRIPTION OF THE EMBODIMENTS

Hereinafter, embodiments of the present invention will be described with reference to the accompanying drawings so that a person skilled in the art to which the present invention pertains can easily carry out the present embodiments. As will be understood by those skilled in the art to which the present invention pertains, the embodiments described below can be modified into various forms without departing from the concept and scope of the present invention. The same or similar parts are denoted using the same reference numerals in the drawings as much as possible.

Terminologies used hereinafter are provided to describe specific exemplary embodiments and are not intended to limit the present invention. Singular expressions used herein include plurals unless they have definitely opposite meanings. The meaning of “including” used in this specification refers to specific characteristics, regions, integers, steps, operations, elements, and/or components, and does not exclude the existence or addition of other specific characteristics, regions, integers, steps, operations, elements, components, and/or groups.

All terminologies including technical terms and scientific terms used hereinafter have the same meanings as those skilled in the art generally understand. Terms defined in dictionaries are construed to have meanings corresponding to related technical documents and the present description, and they are not to be construed as ideal or overly official meanings, if not defined.

Hereinafter, ginseng will be described as an example in the following description. The present invention may be applied to various plants, such as Chamaecyparis obtusa and Phellodendron amurense, including balloon flowers, Codonopsis lanceolata, burdock, and the like, which contain high saponin contents, besides ginseng.

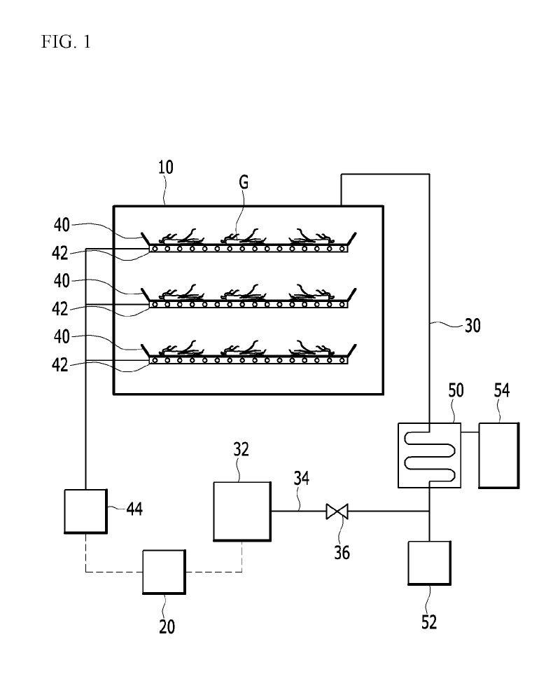

FIG. 1 is a schematic diagram showing a structure of a facility for conversion and extraction of beneficial components in ginseng according to the present embodiment.

As shown in the drawing, a facility of the present embodiment is for heating and drying ginseng under vacuum, and the facility may include a vacuum chamber 10, a decompressing part connected to the vacuum chamber 10 to apply vacuum pressure to the vacuum chamber 10, and a heating part for heating ginseng G placed in the vacuum chamber 10.

The facility of the present embodiment may further include a controller 20 connected to the decompressing part and the heating part, wherein the controller 20 adjusts the vacuum pressure and the heating temperature by controlling the decompressing part and the heating part according to desired conditions.

The vacuum chamber 10 is a sealed structure, and has an accommodation space therein and is isolated from the outside

The decompressing part is connected to the vacuum chamber 10 to apply a vacuum pressure to the vacuum chamber 10. Therefore, the vacuum chamber 10 provides a vacuum pressure which is maintained to be suitable for ginseng accommodated therein by the decompressing part. Accordingly, moisture contained in the ginseng is separated and extracted while the boiling point of ginseng is lowered, so that solids, volatile materials, and moisture can be separated from each other.

Ginseng may be heated in the decompression environment while the boiling point is maintained at 25° C. to 35° C. Therefore, while a predetermined degree of vacuum is maintained, the moisture contained in ginseng can be separated and extracted and even a volatile flavor of ginseng can be collected without modification and influence by the temperature. Since ginseng is dried while boiling is conducted at a boiling point of 25° C. to 35° C., the tissues of ginseng are sterilized and the solids show beneficial components that are visible in red ginseng.

The decompressing part is connected to the vacuum chamber 10 to reduce the inner pressure of the vacuum chamber 10 to a predetermined degree of vacuum pressure and to continuously maintain the vacuum pressure during the drying procedure of the ginseng. The decompressing part may include: a discharge pipe 30 installed at the vacuum chamber 10 to communicate with the inside of the vacuum chamber 10; and a driver 32 connected to the discharge pipe 30 to forcibly discharge inner air of the vacuum chamber 10 to the outside.

The decompressing part is connected to a suction pipe line 34 that is diverged from the front end of the discharge pipe 30. A control valve 36, which opens/closes the suction pipe line or allows the suction pipe line to communicate with air to adjust the vacuum pressure inside the vacuum chamber 10, is installed on the suction pipe line 34. Therefore, when the driver 32 is driven, the suction power is generated through the suction pipe line 34 connected to the discharge pipe 30, and thus the inner air of the vacuum chamber 10 escapes to the outside through the discharge pipe 30, so that the inside of the vacuum chamber 10 is decompressed. The moisture evaporated from ginseng G as well as the inner air of the vacuum chamber 10 is continuously discharged through the discharge pipe 30, and this will be described again later.

The driver 32 may have various structures, such as an apparatus using a vacuum pump or an apparatus using a water injection structure, and any structure may be applied as long as the inner air of the vacuum chamber 10 can be evacuated and the decompression state is continuously maintained.

In the present embodiment, the heating part may include a plurality of loading shelves 40, which are arranged in the vacuum chamber 10 and onto which ginseng G is loaded, and heating units 42 installed at the loading shelves to apply heat to the ginseng G through the loading shelves. The heating units 42 may be composed of heat wires for converting electrical energy into thermal energy. The heating units may employ various heat sources, such as high-temperature steam, besides the heat wires, but are not limited thereto. The heating units 42 are connected to a power supply 44 to receive necessary electric power for applying electrical energy.

The controller 20 adjusts the vacuum pressure inside the vacuum chamber 10 or the heating temperature by the heating units 42 by controlling the control valve 36 of the decompressing part or the power supply 44 of the heating part. The controller 20 can control the vacuum pressure and the heating temperature during the drying procedure of the ginseng to change the kind of beneficial components and the composition ratio of beneficial components extracted from the ginseng.

Thus, the change of extracted constituent components of the ginseng G can be induced for a short time by varying the vacuum pressure and temperature conditions, thereby enhancing the characteristics of ginseng as a raw material. Therefore, various raw materials with different kinds of beneficial components and different composition ratios of beneficial components can be prepared from ginseng.

The facility of the present embodiment may further include a re-treatment part, which is connected to the vacuum chamber 10 to condense and collect the moisture evaporated from the ginseng G. The re-treatment part has a structure in which the moisture is discharged through the discharge pipe 30 and then condensed.

To this end, the re-treatment part includes a condenser 50, which is installed at one side of the discharge pipe 30 communicating with the vacuum chamber 10 to condense the moisture discharged through the discharge pipe 30, and a collection container 52 to which the condensed moisture discharged from the front end of the discharge pipe 30 is collected.

The discharge pipe 30 serves as a pipe line through which the inner air of the vacuum chamber 10 and the moisture discharged from the ginseng G escape. The driver 32 is installed on the suction pipe line 34 that is diverged from the front end of the discharge pipe 30 after passing through the condenser 50. The collection container 52 is installed at the end of the discharge pipe 30. When the driver 32 of the decompressing part is driven, the suction pressure is generated in the discharge pipe 30 and the inner air of the vacuum chamber 10 flows out through the discharge pipe 30. Thus, the moisture evaporated from the ginseng also continuously escapes through the discharge pipe 30 following the flow of the inner air of the vacuum chamber 10. The moisture passing through the discharge pipe 30 is condensed through the condenser 50 installed at one end of the discharge pipe 30.

The condenser 50 is formed as a structure in which the condenser 50 wraps the discharge pipe 30 and cooling water is distributed inside the condenser 50. A cooling unit 54 for distributing cooling water is connected to the condenser 50. Thus, the cooling water cooled by the cooling unit 54 is circulated to the condenser 50 to cool the discharge pipe 30 passing through the condenser. Therefore, the moisture flowing along the discharge pipe 30 is cooled and condensed while passing through the condenser. The collection container 52 in which condensed water is collected is installed at the end of the discharge pipe 30 passing through the condenser 50, so that the condensed water is dropped in the collection container 52.

Here, the vacuum chamber 10 and the collection container 52 are connected to each other through the discharge pipe 30 while a vacuum state is maintained, so that the discharge of the moisture evaporated from the ginseng G to the outside during drying can be completely blocked. Therefore, all the moisture evaporated from the ginseng is collected as condensed water without being externally discharged, and thus the condensed water can be reused in various forms.

Hereinafter, the vacuum drying procedure of the ginseng according to the present embodiment will be described.

In the present embodiment, beneficial ginseng components can be converted and extracted in a short time through a process in which the ginseng is heated and dried under vacuum pressure.

Ginseng is spread and loaded on the loading shelves provided in the vacuum chamber.

In addition, a predetermined degree of vacuum pressure is applied into the vacuum chamber, and the ginseng is heated to a predetermined temperature through the loading shelves. As the ginseng is heated to a high temperature under the vacuum pressure, a tissue change of the ginseng and physical and chemical changes of constituent components of the ginseng are induced.

As the heating of the ginseng is conducted under the vacuum pressure, the moisture from the ginseng tissues is evaporated and the evaporation heat is lost. Thus, even if the ginseng is directly heated at a high temperature through the loading shelves, the ginseng can be dried while the carbonization of ginseng tissues is prevented. That is, the evaporation occurs on a contact surface of the ginseng, which is in direct contact with the loading shelves, so that the temperature of the loading shelves is maintained to be low by the evaporation heat of the water vapor evaporating from ginseng even if the temperature of the loading shelves is increased. Therefore, the carbonization of ginseng can be prevented even while the drying rate is increased. In addition, physical changes of constituent components of ginseng are caused by high decompression conditions and a high-temperature environment.

In the present embodiment, the vacuum pressure for ginseng may be −95 kPa to −98 kPa. Optimal extraction efficiency of beneficial components can be obtained under the above range of vacuum pressure. For example, if the vacuum pressure is lower than −95 kPa, the boiling point increases, and the extraction efficiency may be influenced by the temperature. If the vacuum pressure is higher than −98 KPa, the effect of increasing the extraction efficiency of beneficial components is hardly observed, the vacuum pressure is difficult to maintain due to the evaporation amount of ginseng generated during the extraction procedure, and a large-capacity facility is required.

In the present embodiment, the heating temperature for ginseng may be 60° C. to 120° C. The ginseng heating temperature is controlled by the heating units installed at the heating units, thereby gently increasing the temperature from a low temperature to a high temperature. The optimal extraction efficiency of beneficial components can be obtained in the above heating temperature range. For example, if the heating temperature is higher than 120° C., the deformation of ginseng by radiant heat may occur even though the drying time is shortened. If the heating temperature is lower than 60° C., the drying time may be longer and thus the drying yield may be reduced.

In addition, the treatment time for heating and drying ginseng under the vacuum pressure may be 5 hours to 24 hours.

Optimal extraction efficiency of beneficial components can be obtained within the above range of treatment time. For example, if the treatment time is longer than 24 hours, carbonization partially occurs, and if the treatment time is shorter than 5 hours, a difference in the degree of drying may occur, causing a deviation in the degree of drying of solids.

The heating temperature and the treatment time of ginseng may be adjusted depending on the size of the ginseng. For example, the heating temperature and the treatment time can be shortened by 20 to 30% in smaller and thinner ginseng compared to larger and thicker ginseng.

The heating temperature and the heating time of ginseng may be adjusted according to the purpose of use of the extract. That is, the extraction efficiency of a desired material can be increased and the composition ratio of beneficial components can be varied by changing the heating temperature and heating time conditions of ginseng.

In the present embodiment, when the heating temperature of ginseng is set to 100° C. to 120° C. and the treatment time is set to 8 hours to 12 hours, saponins (e.g., G-Rg3) inherent to ginseng can be enriched and high-boiling point components can be efficiently extracted.

In addition, when the heating temperature of ginseng is set to 60° C. to lower than 100° C. and the treatment time is set to 6 hours to 15 hours, the extraction efficiency of non-saponin-based active materials can be increased and low-boiling point components (e.g., panacene and β-sesquiterpenoids) can be extracted or removed more efficiently.

As described above, the composition ratio of desired beneficial components can be varied by changing the treatment conditions. Thus, the contents of specific ginseng beneficial components suitable for the purpose of use of the extract can be increased and a difference in the constituent components of the produced ginseng processed product can be induced.

Therefore, ginseng dried products having various beneficial components with changed chemical structures can be produced through the present embodiment, so that raw materials in various application fields can be produced through a difference of the changed composition ratio.

The moisture generated in the drying of ginseng during the ginseng heating procedure can be collected through condensation. The moisture generated from ginseng during the heating and drying procedure is discharged through the discharge pipe 30 connected to the vacuum chamber 10, and condensed and separated passing through the condenser. In addition, the condensed water is collected in a separate collection container connected to the end of the discharge pipe 30.

The water vapor generated from ginseng during the heating and drying procedure is not discarded but is all collected, and thus can be used through retreatment. The water vapor contains various aromatic materials evaporated from ginseng, and thus the collection of the water vapor can be developed into various products.

As such, ginseng can be effectively dried through the present drying method, and additionally, the condensed water can be further obtained, so that additional products can be developed.

### EXAMPLES

In the present example, beneficial components were extracted by heating and drying ginseng under vacuum pressure as mentioned above.

In a comparative example, ginseng was dried in the high-temperature and high-pressure environment as in the prior art, and such a drying procedure was repeated nine times to extract beneficial components.

TABLE 1

| | | Extracted | |
|---|---|---|---|
| | | component | |
| Classification | Treatment time | (ginsenosides) | Content |
| Example | 24 | Rg1 | 3.52 |
| | hours | Re | 5.31 |
| | | Rf | 0.97 |
| | | Rg2(S) | 0.03 |
| | | Rg2(R) | 0.28 |
| | | Rb1 | 5.37 |
| | | Rb2 | 1.97 |
| | | Rc | 0.67 |
| | | Rb3 | 0.28 |
| | | Rd | 0.11 |
| | | Total | 18.51 |
| Comparative | 200 | Rg1 | 2.35 |
| Example | hours | Re | 2.48 |
| | | Rf | 1.47 |
| | | Rb1 | 4.56 |
| | | Rc | 3.21 |
| | | Rb3 | 0.42 |
| | | Rd | 0.82 |
| | | Total | 4.45 |

As shown in Table 1 above, it can be confirmed that 200 hours were required for the treatment time due to nine repetitions of the process in the comparative example in which steaming and drying were repeated in the conventional high-temperature and high-pressure environment, whereas heating and drying were conducted under the vacuum pressure in the present example, so that the treatment time can be shortened to within 24 hours.

In addition, even though ginseng was treated for a short time of within 24 hours in the present example, when compared with the comparative example, the present example can obtain effects of inducing changes in desired beneficial components, shortening the extraction time of beneficial components contained in the dried product, and increasing the extract contents.

In a case where ginseng was steamed and dried as in the comparative example, the preparation yield of red ginseng obtained by steaming and drying once was about 30%, and the yield of black ginseng obtained by repeatedly steaming and drying was lower than such a yield value. The reason is that constituent components of ginseng leaked out due to repeated steaming and drying. It is known that the steaming and drying of ginseng for preparation of red ginseng increases the contents of acidic polysaccharides and induces structural changes of ginsenosides, thereby increasing the diversity of ginsenosides, thus improving medical efficacy.

Therefore, as shown in the present example, a dried product having the same medical effect as in black ginseng can be effectively prepared in a short time without leakage of constituent components of ginseng by drastically shortening the treatment time for ginseng.

In addition, the heating and drying process under the vacuum pressure as in the present example can be applied to various crops besides ginseng in order to change beneficial components and increase extraction efficiency. For example, the conventional extraction of phytoncides from Japanese cypress was conducted such that the components isolated through the heated steam were condensed and collected or the components were extracted using an organic solvent, but the application of the present embodiment can exclude the use of an organic solvent, and separate and extract essential oil components having different boiling points by varying the temperature and vacuum degree, thus increasing extraction efficiency. Therefore, the treatment time can be shortened and the extraction efficiency can be increased by applying the present embodiment to the extraction and modification of beneficial components, such as aromatic components, of plants.

As set forth above, the illustrative embodiments of the present invention have been shown and described, but various modifications and alternative embodiments may be made by those skilled in the art. Such modifications and other embodiments could be made without departing from the scope of the present invention as defined by the appended claims.

<Description of symbols>

| | |
|---|---|
| 10: vacuum chamber | 20: controller |
| 30: discharge pipe | 32: driver |
| 34: suction pipe line | 36: control valve |
| 40: loading shelf | 42: heating unit |
| 44: power supply | 50: condenser |
| 52: collection container | 54: cooling unit |

1. A method for conversion and extraction of beneficial components in ginseng, the method comprising:
a vacuum step of feeding ginseng into a vacuum chamber and applying a vacuum pressure thereinto; and
a treatment step of heating and drying the ginseng under the vacuum pressure.

2. The method of claim 1, further comprising a collecting step of condensing and collecting moisture generated during the drying of the ginseng in the treatment step.

3. The method of claim 1, wherein in the vacuum step, the vacuum pressure is −95 kPa to −98 kPa.

4. The method of claim 3, wherein in the treatment step, the temperature for the heating is 60° C. to 120° C.

5. The method of claim 4, wherein the treatment step is carried out for 5 hours to 24 hours.

6. The method of claim 2, wherein in the vacuum step, the vacuum pressure is −95 kPa to −98 kPa.

7. The method of claim 6, wherein in the treatment step, the temperature for the heating is 60° C. to 120° C.

8. The method of claim 7, wherein the treatment step is carried out for 5 hours to 24 hours.
